(19) United States
(12) Patent Application Publication
(10) Pub. No.: US 2015/0257743 A1
(43) Pub. Date: Sep. 17, 2015

(54) PERICARDIAL ACCESS DEVICE

(71) Applicants: James W. Meador, Houston, TX (US); Debra J. Lucas, San Diego, CA (US)

(72) Inventors: James W. Meador, Houston, TX (US); Debra J. Lucas, San Diego, CA (US)

(21) Appl. No.: 14/214,857

(22) Filed: Mar. 15, 2014

(51) Int. Cl. A61B 17/00; A61B 17/34

(52) U.S. Cl. CPC A61B 17/00234; A61B 17/3403; A61B 17/3423; A61B 2017/00243; A61B 2017/3405; A61B 2017/347; A61B 17/3478

(57) ABSTRACT

A device for percutaneously accessing the pericardial space has a tubular body wall enclosing a lumen. The wall ends in a tip that has an inferior aperture in fluid communication with the lumen. The tip surrounds and roofs over the aperture to form an chamber above the aperture, The portion of the tip over the aperture has a plurality of grooves in fluid communication with the lumen. The device includes a hollow body with a piercing end moveable in the first tubular body to extend into the chamber. A vacuum applied by the device to the chamber draws a bleb of pericardium into the chamber where it can be safely pierced by the piercing body to fain access to the pericardial space.

### CROSS-REFERENCE TO RELATED APPLICATIONS

This application claims under 35 U.S.C. §119(e) the benefit of U.S. Provisional Application 61/787,326 filed on Mar. 15, 2013, the content of which is incorporated in its entirety.

### STATEMENT REGARDING FEDERALLY SPONSORED RESEARCH AND DEVELOPMENT

Not Applicable

### BACKGROUND OF THE DISCLOSURE

1. Field of Disclosure

This invention relates to methods and apparatus for accessing the pericardial space for medical treatment of the heart.

2. Background

Knowledge of the pericardium dates back to the time of Galen (129-200 A.D.) the Greek physician and anatomist who gave the pericardium its name. The pericardial sac surrounds the heart like a glove enfolds a hand, and the pericardial space is naturally fluid-filled. The normal pericardium functions to prevent dilatation of the chambers of the heart, lubricates the surfaces of the heart, and maintains the heart in a fixed geometric position. It also provides a barrier to the spread of infection from adjacent structures in the chest, and prevents the adhesion of surrounding tissues to the heart. See generally, Holt J P: The normal pericardium, Amer J Cardiol 26:455, 1970; Spodick D H: Medical history of the pericardium, Amer J Cardiol 26:447, 1970. The normal pericardial space is small in volume and the fluid film within it is too thin to functionally separate the heart from the pericardium. It has been observed that when fluid is injected into the pericardial space it accumulates in the atrioventricular and interventricular grooves, but not over the ventricular surfaces.

Pericardiocentesis, or puncture of the pericardium, heretofore has been indicated for (1) diagnosis of pericardial disease(s) by study of the pericardial fluid, (2) withdrawal of pericardial fluid for the treatment of acute cardiac tamponade, (3) infusion of therapeutic agents for the treatment of malignant effusion or tumors and (4) infusion of stem cells for therapeutic treatment of the heart At present, intrapericardial injection of drugs is clinically limited to the treatment of abnormal pericardial conditions and diseases, such as malignant or local pericardial effusions and tumors.

Intrapericardial drug delivery has not been clinically utilized for heart-specific treatments where pericardial pathology is normal, because the pericardial space is normally small and very difficult to access without invasive surgery or risk of cardiac injury by standard needle pericardiocentesis techniques. The pericardiocentesis procedure is carried out by experienced personnel in the cardiac catheterization laboratory, with equipment for fluoroscopy and monitoring of the electrocardiogram. Electrocardiographic monitoring of the procedure using the pericardial needle as an electrode is commonly employed. Complications associated with needle pericardiocentesis include laceration of a coronary artery or the right ventricle, perforation of the right atrium or ventricle, puncture of the stomach or colon, pneumothorax, arrhythmia, tamponade, hypotension, ventricular fibrillation, and death. The complication rates for needle pericardiocentesis are increased in situations where the pericardial space and fluid effusion volume is small (i.e., the pericardial size is more like normal).

The described invention is a mechanism to achieve pericardial capture, puncture and therefore fluid access to the epicardium to undertake a variety of therapies and is an improvement over the devices described in patents: U.S. Pat. Nos. 5,827,216; 5,900,433; 6,162,195 and 6,666,844, the contents of which are incorporated in their entirety for background.

### BRIEF DESCRIPTION OF THE DRAWINGS

In the following detailed description of exemplary embodiments, reference is made to the accompanying drawings, which form a part hereof and in which are shown by way of illustration examples of exemplary embodiments with which the invention may be practiced. In the drawings and descriptions, like or corresponding parts are marked throughout the specification and drawings with the same reference numerals. The drawings are not necessarily to scale. Certain features of the invention may be shown exaggerated in scale or in somewhat symbolic or schematic form and some details of conventional elements may not be shown in the interest of clarity and conciseness. Referring to the drawings:

FIG. 1 is a general view of one end of one embodiment of the present invention.

FIG. 2A is an axial centerline sectional schematic view of an embodiment of an introducer tip of the present invention.

FIG. 2B is a front partially cross sectional schematic view of the tip of FIG. 2A.

FIG. 3 is an axial centerline sectional schematic view of the tip of FIG. 2A showing a needle and guidewire.

FIG. 4A is a side elevational partially axial sectional view of another embodiment of an introducer tip of the invention.

FIG. 4B is side elevational partially axial sectional schematic view of the embodiment of FIG. 4A.

FIG. 4C is an end view of the embodiment of FIG. 4A as shown deployed in FIG. 4B.

FIG. 4D is a bottom view of the embodiment of FIG. 4A as shown deployed in FIG. 4B showing a deployed needle.

FIG. 5A is an axial centerline sectional schematic view of another embodiment of an introducer tip of the invention.

FIG. 5B is a view of the proximal end of the invention and a view of associated electronics.

FIG. 6A is a view of the proximal end of another embodiment of the invention.

FIG. 6B is a schematic of another embodiment of the invention with the tip in an upward position.

FIG. 6C is a schematic of another embodiment of the invention with the tip in a downward position.

FIG. 7A is an axial centerline sectional schematic view of another embodiment of an introducer tip the invention.

FIG. 7B is an axial centerline sectional schematic view of the tip of FIG. 7A showing an extended probe.

FIG. 7C is an axial centerline sectional schematic view of the tip of FIG. 7A showing an attached pericardium.

FIG. 7D is an axial centerline sectional schematic view of the tip of FIG. 7A showing a pierced pericardium and deployment of a guidewire.

### DETAILED DESCRIPTION OF EMBODIMENTS

Specific details described herein, including what is stated in the Abstract, are in every case a non-limiting description and exemplification of embodiments representing concrete ways in which the concepts of the invention may be practiced. This serves to teach one skilled in the art to employ the present invention in virtually any appropriately detailed system, structure or manner consistent with those concepts. Any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments that may or may not be given therewith or elsewhere in the specification and all such embodiments are intended to be included within the scope of that term or terms. Language designating such non-limiting examples and illustrations includes, but is not limited to: “for example,” “for instance,” “e.g.,” “in an embodiment,” “in an exemplary embodiment.”

Reference throughout this specification to “an embodiment” means that a particular feature, structure, or characteristic described in connection with the embodiment is included in at least one exemplary embodiment of the present invention. Thus, the appearances of the phrase “in an exemplary embodiment” in various places throughout this specification are not necessarily all referring to the same embodiment. Furthermore, the particular features, structures, or characteristics may be combined in any suitable manner in one or more embodiments. It will be seen that various changes and alternatives to the specific described embodiments and the details of those embodiments may be made within the scope of the invention. It will be appreciated that one or more of the elements depicted in the drawings can also be implemented in a more separated or integrated manner, or even removed or rendered as inoperable in certain cases, as is useful in accordance with a particular application. Because many varying and different embodiments may be made within the scope of the inventive concepts herein described and in the exemplary embodiments herein detailed, it is to be understood that the details herein are to be interpreted as illustrative and not as limiting the invention to that which is illustrated and described herein.

As used herein, the terms “comprises,” “comprising,” “includes,” “including,” “has,” “having” or any other variation thereof, are intended to cover a non-exclusive inclusion. For example, apparatus that comprises a list of elements is not necessarily limited to only those elements but may include other elements not expressly listed or inherent to such process, article, or apparatus. Further, unless expressly stated to the contrary, “or” refers to an inclusive or and not to an exclusive or. That is, unless otherwise indicated, the term “or” is generally intended to mean “and/or”. For example, a condition A or B is satisfied by any one of the following: A is true (or present) and B is false (or not present), A is false (or not present) and B is true (or present), and both A and B are true (or present).

As used herein, a term preceded by “a” or “an” (and “the” when antecedent basis is “a” or “an”) includes both singular and plural of such term (unless in context the reference “a” or “an” clearly indicates only the singular or only the plural). Thus the use of the word “a” or “an” may mean “one,” but it is also consistent with the meaning of “at least one” and “one or more than one.”

As used in the description herein, the meaning of “in” includes “in” and “on” unless the context clearly dictates otherwise.

In addition, as used herein, the phrase “connected” means joined to or placed into communication with, either directly or through intermediate components.

The various directions such as “upper,” “top”, “lower,” “bottom”, “back,” “front,” “transverse,” “vertical”, “horizontal,” “length,” “Longitudinal,” “width,” “laterally,” “forward,” “rearward” and so forth used in the detailed description of exemplary embodiments are made only for easier explanation in conjunction with the drawings. The components may be oriented differently while performing the same function and accomplishing the same result as the exemplary embodiments herein detailed embody the concepts of the invention, and such terminologies are not to be understood as limiting the concepts which the embodiments exemplify.

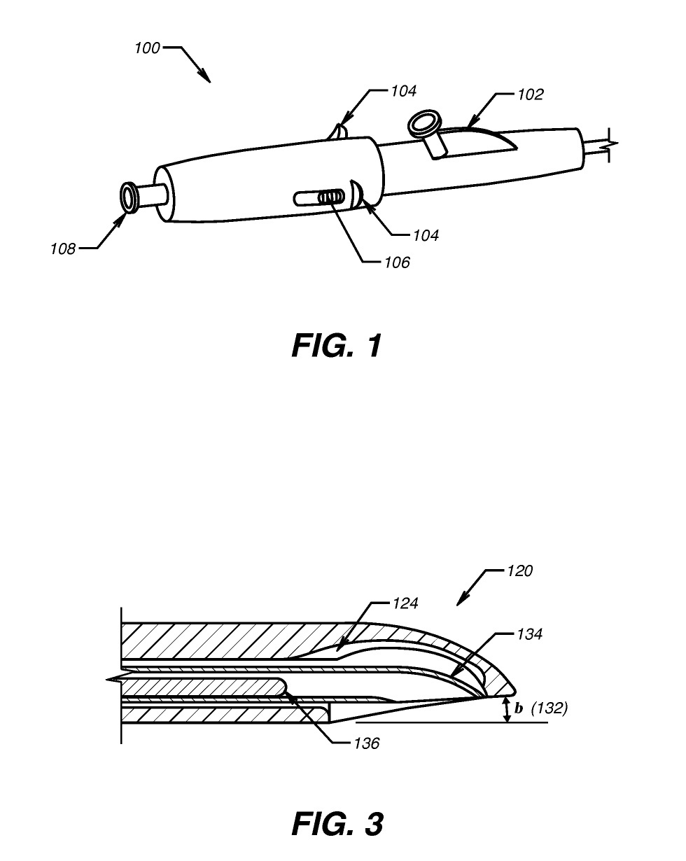

The current invention, 100 is a next generation device of the prior art device described in U.S. Pat. Nos. 5,827,216; 5,900,433; 6,162,195 and 6,666,844, the contents of which are incorporated in their entirety. Several improvements over the prior art device is shown in FIG. 1. Notably there is a finger grip 102 on the edge by the vacuum connection. This orients the hand more intuitively. There are push tabs 104 on the sides of the device which also orient the hands. The needle is locked within the housing to prevent premature advancement which will inhibit piercing of the pericardium. A locking release button 106 on the side of the unit will permit unlocking and advancement of the needle when ready by the user. The proximal end of the device is a female leur device 108 instead of the integral Tuohy found on the prior art device. This still permits a Tuohy to be attached and a stopcock so that fluids can be injected into the device through the needle, as desired. A principal change is changing the standard beveled needle which used to require rotation, to a Tuohy which does not require rotation to pierce the pericardium and keep the needle tip low in profile and near the same plane as the bottom of the needle shaft. This permits optimum piercing of the pericardium, as explained later.

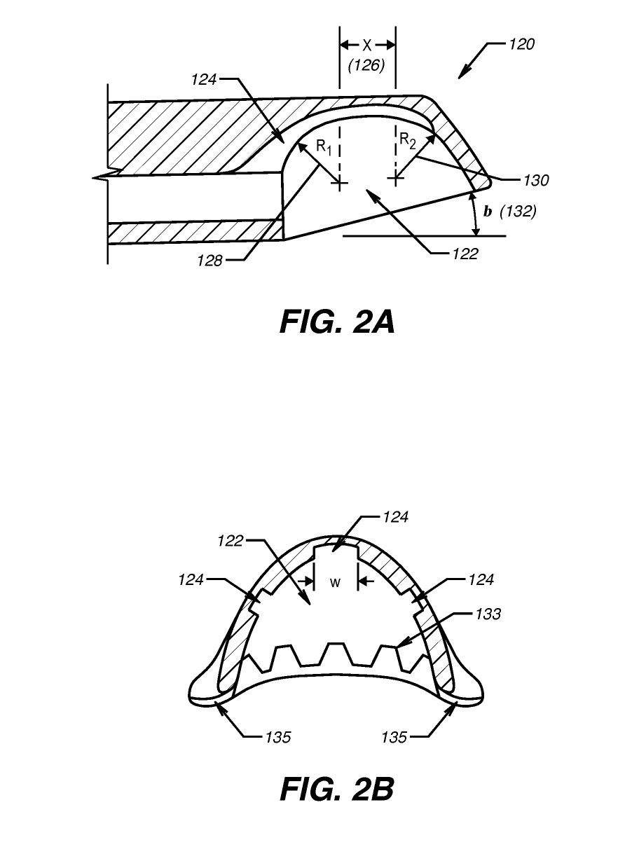

The invention 100 also has changes in the tip 120 and the bleb chamber 122, as shown in FIG. 2A. As vacuum is applied, the pericardium is drawn up into the bleb chamber 122. To ensure that the pericardium is completely drawn into the bleb chamber, small channels are molded into the tip (vacuum fingers 124) so that with a partially drawn-in pericardium in the proximal part of the bleb chamber, there still is vacuum to the distal end of the bleb chamber to ensure that the pericardium is fully drawn into the bleb chamber. An end view of the vacuum fingers or grooves is seen in FIG. 2B. FIG. 2B also shows a lower portion of tip 120 provided with reliefs 133 in the tip to effect a serration to help scrub pericardial fat from the surface of the pericardium. Also, as shown in FIG. 2B, a flexible area 135 is provided around the tip so that the distal portion of the tip can flexibly adapt to the pericardial/heart geometry without being rigid.

While the current bleb chamber 122 of the present invention and the prior art device are hemispherical, there is no reason that they need to be. An obround chamber is fully possible, shown in FIG. 2. As “X” 126 goes to zero, then the chamber becomes a hemisphere if R1 (128)=R2 (130). The vacuum fingers 124 become increasingly important as X (126)>0 due to the direction of the vacuum pressure.

A goal of the present invention is to safely puncture the pericardial sac and be in fluid communication with the heart to deliver therapies. Currently, the bottom of the plastic tip of the device is parallel to the axis of the needle. As long as the tip of the needle 134 does not protrude outside a line between the proximal and distal part of the bleb chamber, then the heart cannot be lacerated. Consequently, the angle β (132) can be confirmed, as shown in FIGS. 2A and 3. Then β represents the angle from the edge of the distal portion of the bleb chamber to the distal tip of the needle and to the proximal edge of the bleb chamber so the needle cannot touch the heart.

The Tuohy needle 134 is an advantage over the previous straight beveled needle, as the tip of the Tuohy is bent so that the tip is on a plane with the lower part of the needle tubing. Obviously the exit of the needle needs to be pointed down (towards the heart) for maximum benefit for addition of fluid, removal of fluid, and for placing a guidewire 136, which is resident within the needle 134, as shown in FIG. 3. Using a conventionally beveled needle as in the prior art device, the needle was rotated so that the bevel was in the “up” position (away from the heart) to pierce the pericardium and then rotated so that the bevel was “down” (toward the heart) for fluid aspiration/delivery and for guidewire delivery. The Tuohy removes the need for needle rotation and therefore makes the device simpler to operate.

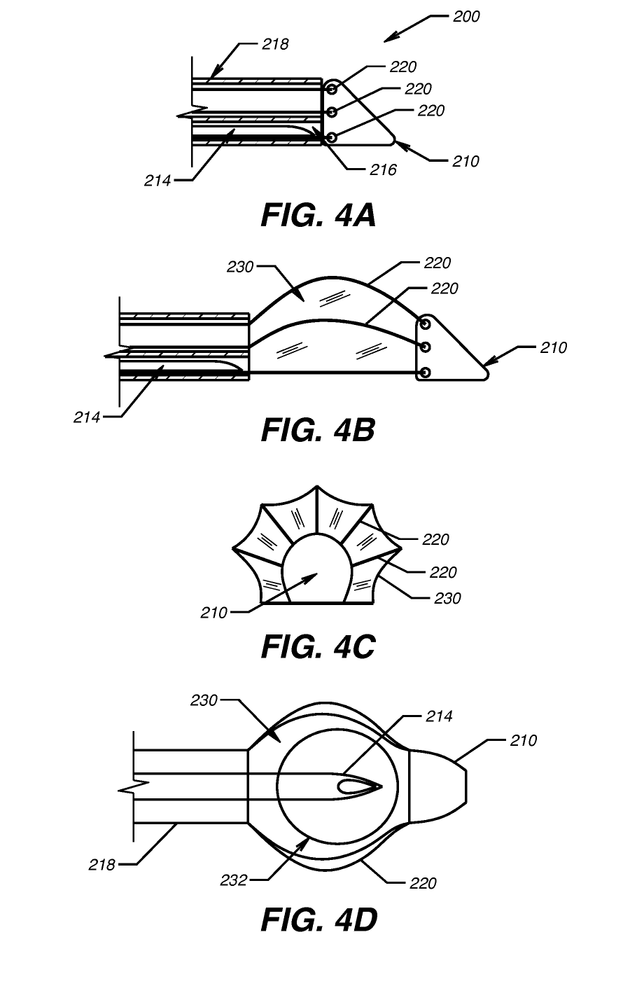

The prior art device had a solid tip and “bleb” chamber for the vacuum introduction of the pericardium into the instrument. The size of the tip 120 determined the size of the incision into the patient and the diameter of the dilator/sheath. In this circumstance, the smaller the incision/introducer, the better it is for the patient. However, a thickened pericardium, the choice of larger needle sizes for lancing the pericardium, and entering the pericardium for heart access would argue for larger tips 120. This embodiment permits both, by permitting smaller incisions and instrument introduction while deployable and expansive tip. FIG. 4 (A-D) illustrates the tip of this embodiment device, generally noted as 200. FIG. 4A illustrates the device as introduced into the patient. FIG. 4B illustrates the apparatus 200 extended. FIG. 4C illustrates the end view of the apparatus 200. FIG. 4D illustrates the bottom view of the apparatus 200 with the needle 214 deployed.

Notable is the nose of the device 210, which has a variety of struts 220 attached thereto. The struts 220 are preferably made of Nitenol, a memory metal but can be made of any material which can hold its shape. The needle 214 is housed in a tube 216 and the apparatus is housed in a sheath 218. The deployed struts 220 expand to form a canopy, as illustrated in FIG. 4B. Flexible housing 230 is sealed to the inside of the rigid sheath 218 and is formed into shape by struts 220, once deployed. FIG. 4C illustrates an end view of the device showing the formation of flexible housing 230. After vacuum is applied within sheath 218, the pericardium will be pulled into the flexible housing 230, braced by struts 220. FIG. 4D illustrates the underside of the device and has a circular opening 232. When vacuum is deployed within sheath 218, the circular opening will seal against the pericardium and permit a pericardial bleb to be drawn into the geometry formed by struts 220 and flexible housing 230. At that point, the needle 214 is advanced and pierces the pericardium. Once pierced, the interior of the needle 214 is in communication with the inside of the pericardium and therefore the exterior of the heart. Fluid from the pericardial space can be withdrawn, fluid can be added through the needle into the pericardial space, and/or a guidewire can be deployed via the needle into the pericardial space. At the appropriate time, the needle 214 may be pulled back into the tube 216, and the struts pulled back into the sheath 218 and the device again resembling the configuration of FIG. 4A. In this low profile configuration, the device can be removed from the patient.

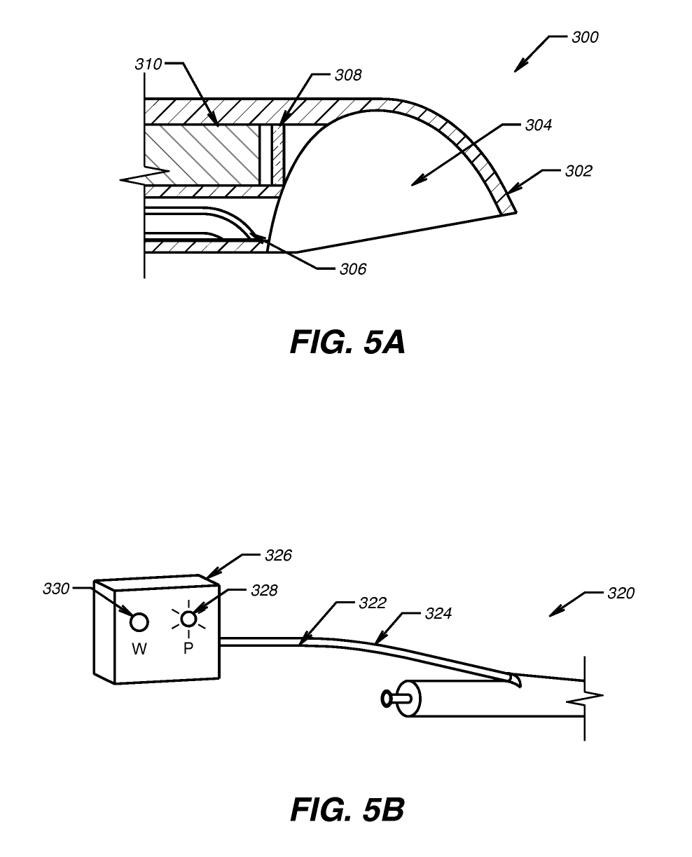

FIG. 5A illustrates another embodiment of the present invention. The distal tip of the device is generally shown as 300, with the housing shown as 302 and the bleb chamber 304. It is always worthwhile to have positive feedback that the pericardium has indeed been captured into the bleb chamber 304 so that the needle 306 may advance into the bleb chamber 304, pierce the captured pericardium, and be in fluid communication with the outside of the heart. To help effect this feedback, a miniature CCD camera 310 is placed in the tip 300. Window 308 may be suitable as a filter or other optical mechanism to assist the camera 310 in transmitting the image within the bleb chamber 304 to the physician or other individual outside the patient. This can provide confirmation that the pericardium has indeed been captured into the bleb chamber 304 and it is appropriate to advance the needle 306 to pierce the pericardium captured within the bleb chamber 304 using vacuum.

The filter 308 and the camera 310 may be coupled with an electronics system outside the device, as illustrated in FIG. 5B. The device is shown generally as 320. Wires (or wireless communications), shown as 322 and 324, electronically connect the device 320 to the outside electronics, generally shown as 326. The optical camera/detector 310 aided by software and/or the physical window/filter 308 detects the presence of the pericardial sac being pulled into the bleb chamber 304 by applied vacuum. The optical characteristics of that event will cause a change in the output of the camera/detector 310 and this signal change can be processed by the electronics 326 in a very straightforward threshholding manner. This will change indicator lights from “W” (wait to advance the needle) to “P” (advance to puncture the pericardium) as the system will have determined, after the application of vacuum, if the pericardium has been captured into the bleb chamber 304. In this manner, the operators of the device have the operational feedback they need to more accurately. Both of these approaches are represented in FIGS. 5A and 5B.

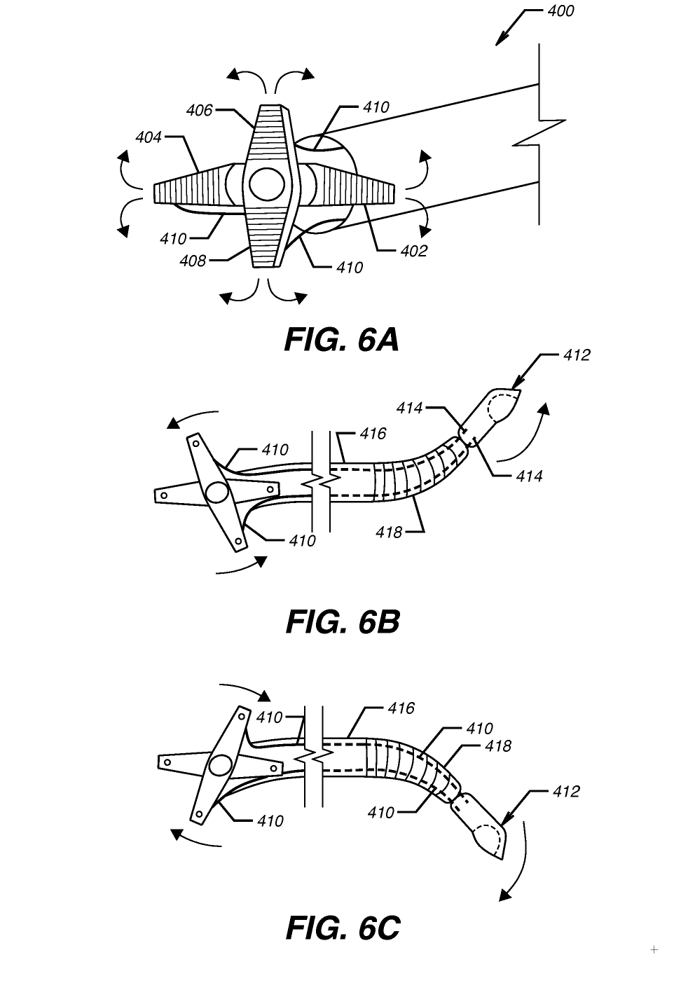

FIG. 6 illustrates another embodiment of the present invention and is about control over the tip. The general device is illustrated as 400. The proximal end contains control paddles 402, 404, 406 and 408, as shown in FIG. 6A. Paddles 402 and 404 are connected together as are paddles 406 and 408, so when paddle 402 is depressed inward, paddle 404 extends outward and vice versa. The same is true for paddles 406 and 408, so when paddle 408 is depressed inward, paddle 406 moves outward and vice versa. FIG. 6B illustrates that control cables 410 are connected to each of the paddles and to the respective portion of the tip 412, shown in two of four places 414—two to produce vertical movement with the paddles and two to produce lateral movement with the paddles. The shaft of the device includes the rigid portion 416 and a flexible portion 418. In use, moving paddle 402 inward, as shown in FIG. 6B causes the control wire 410 attached to the paddle 402 to tighten, and the control wire 410 attached to the other paddle 404 to have slack. The resultant motion moves the tip upward, as shown. FIG. 6C illustrates the reverse function and moving paddle 404 inward causes paddle 402 to move outward and causes the tip 412 to move downward. Analogously, moving paddles 406 and 408 inward/outward causes the tip 412 to move laterally. These paddle pairs work independently so that the tip 412 may by gyrated into a combination of vertical and lateral positions as desired by the operator and using said paddles.

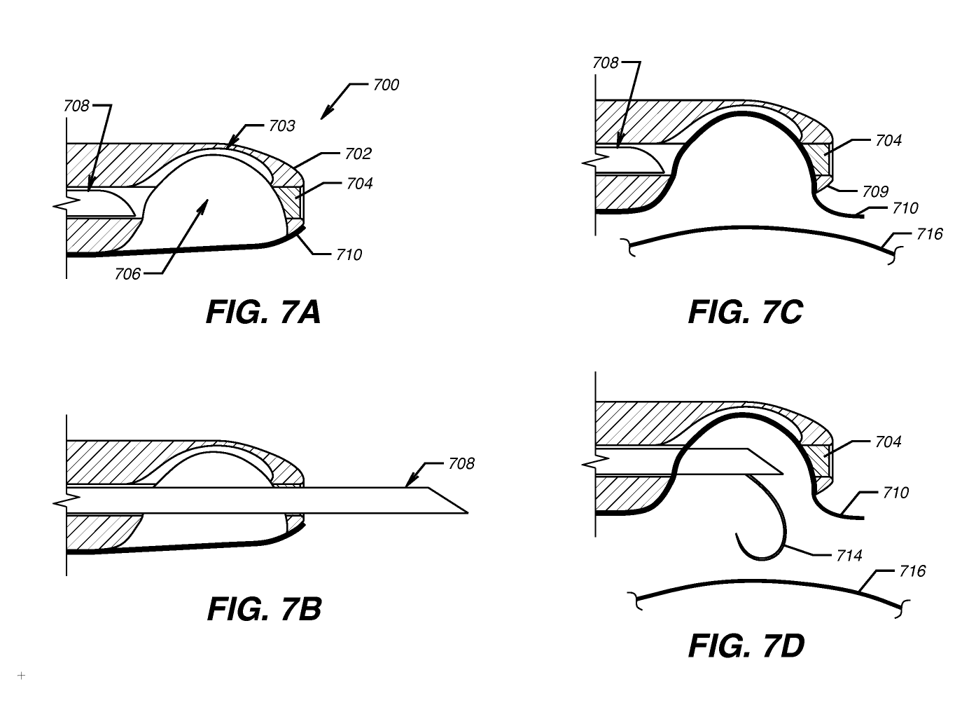

Another embodiment uses the concept of a tunneling rod. This is a Tuohy needle which may eliminate the need for a sheath/dilator. In this embodiment shown in FIG. 7 (A-D), the distal portion of the tip is shown generally as 700 in FIG. 7A, with an overmolded septum 704 and vacuum fingers 703. The lower portion of tip 709 also has a lateral geometry like a serration to help scrub pericardial fat from the surface of the pericardium. After the incision in the body is made, the Tuohy needle 708 is extended through the septum 704, as illustrated in FIG. 7B. The needle and the slender shape of tip 702 may make the insertion easier and may reduce or eliminate the need for a sheath/dilator. Once in place, the needle is retracted, as shown in FIG. 7C. The septum 704 reseals the tip 700. Vacuum is applied and the pericardium 710 is drawn into the bleb chamber 706. FIG. 7D illustrates the needle 708 is advanced into the bleb chamber 706 and it pierces the pericardium 710. The needle can add and/or remove material and/or places an internal guidewire 714 as shown, to give therapy to the heart 716.

Overmolding can also provide other advantages, such as an extended sealing area around the lower edge of the bleb chamber for more efficient pericardial access and capture. It can also provide a flexible area around the tip so that the distal portion of the tip can flexibly adapt to the pericardial/heart geometry without being rigid.

It can be seen that most to all of these embodiments can be combined into a pericardial access device providing a variety of advantages and features.

The above disclosed subject matter is to be considered illustrative, and not restrictive, and the appended claims are intended to cover all modifications, enhancements, and other embodiments that fall within the true scope of the present invention, which to the maximum extent allowed by law, is to be determined by the broadest permissible interpretation of the following claims and their equivalents, unrestricted or limited by the foregoing detailed descriptions of exemplary embodiments of the invention.

1. An apparatus for percutaneously accessing the pericardial space, comprising:
A first tubular body having a substantially cylindrical wall enclosing a lumen in the body, said wall ending distally in a tip, the tip having an inferior aperture in fluid communication with said lumen, said tip surrounding and roofing over said aperture to form an chamber above said aperture, said tip over said aperture having a plurality of grooves in fluid communication with said lumen, and
a hollow body with a piercing end moveable in said first tubular body to extend into said chamber.

2. The apparatus of claim 1 in which a distal extent of said tip terminates higher than a proximate portion of the wall adjacent where said lumen communicates with said aperture, said tip around said aperture angling upwardly to said distal extent.

3. The apparatus of claim 1 in which said distal extent of said tip is serrated.

4. The apparatus of claim 1 in which said tip above said distal extend includes a resealable septum pierceable by said piercing end of said moveable hollow body.

5. The apparatus of claim 1 in which said first tubular body includes a second lumen opening into said chamber, said second lumen housing a capacitance charge device camera and a window proximate the chamber interposed between the camera and the chamber, said window sealing the second lumen from the chamber.

6. The apparatus of claim 5, further comprising an instrument electronically connected to said camera for determining a change in optical characteristics output by said camera indicating that the chamber is occupied.

7. The apparatus of claim 1 wherein said tip includes a nose and said first tubular body except for said nose resides inside a tubular sheath leaving a space between the sheath and the first tubular body, said space containing a collapsed fabric and a plurality of longitudinally arranged bowable struts fitted to said fabric around lateral and superior sides of said first tubular body, said struts and web being proximately anchored inside said sheath and distally connected to said nose, whereby on extension of said tip from said sheath said struts bow out and support said fabric to form a canopy over said aperture.
